(19) United States
(12) Patent Application Publication
(10) Pub. No.: US 2010/0190827 A1
(43) Pub. Date: Jul. 29, 2010

(54) POLYMORPHIC FORM OF RIMONABANT, METHOD FOR PREPARING IT AND PHARMACEUTICAL COMPOSITIONS CONTAINING IT

(75) Inventors: Alain ALCADE, Toulouse (FR); Gilles Anne-Archard, Toulouse (FR); Corinne Gavory, Montbazin (FR); Olivier Monnier, Villeveyrac (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/259,701

(22) Filed: Oct. 28, 2008

Related U.S. Application Data
(63) Continuation of application No. 10/494,211, filed on Oct. 12, 2004, now abandoned.

(30) Foreign Application Priority Data
Nov. 8, 2001 (FR) 01/14579

(51) Int. Cl. A61K 31/454; C07D 401/12; A61P 3/04

(52) U.S. Cl. 514/326; 546/211

(57) ABSTRACT

The present invention relates to a novel crystalline polymorph of rimonabant, its method of preparation and the pharmaceutical compositions containing this novel polymorph.

This application is a continuation of U.S. Application No. 10/494,211, filed Oct. 12, 2004, now pending, which was the National Stage of International application No. PCT/FR02/03,765, filed Nov. 4, 2002, both of which are incorporated herein by reference in their entirety; which claims the benefit of priority of French Patent Application No. 01/14,579, filed Nov. 8, 2001.

The present invention relates to a novel polymorph of N-piperidino-5-(4-chlorophenyl)-1-(2,4-dichlorophenyl)-4-methyl-3-pyrazolecarboxamide and a method for its preparation. More particularly the invention relates to the preparation of this polymorph called form II and to pharmaceutical compositions containing it.

### BRIEF DESCRIPTION OF THE DRAWINGS

FIG. 1 is an infrared spectrum of Form I of rimonabant.

FIG. 2 is an infrared spectrum of Form II of rimonabant.

FIG. 3 is a X-ray diffractogram of Form I of rimonabant.

FIG. 4 is a X-ray diffractogram of Form II of rimonabant.

FIG. 5 is a comparison between the theoretical X-ray powder diffractogram (simulated from the single-crystal) and that obtained experimentally (experimental at the top/theoretical at the bottom).

N-Piperidino-5-(4-chlorophenyl)-1-(2,4-dichlorophenyl)-4-methyl-3-pyrazolecarboxamide whose international nonproprietary name is rimonabant is an antagonist of the CB₁ cannabinoid receptors, which was described for the first time in European patent EP 0 656 354. The method claimed in this patent allows the preparation of rimonabant in crystalline form which will be called form I. It has now been found that rimonabant can exist in various polymorphic crystalline forms which differ from each other in their stability, in their physical properties, in their spectral characteristics and in their method of preparation.

Thus, the subject of the present invention is a novel polymorphic form of rimonabant, called form II, it also relates to methods for preparing rimonabant in its polymorphic form II, and pharmaceutical compositions containing the said form II.

European patent EP 0 656 354 makes no reference to the existence of specific polymorphic forms of rimonabant. In this patent, it is disclosed that the compound is isolated according to conventional techniques; more precisely, according to the embodiments exemplified, the product is obtained after crystallization from isopropyl ether or by cooling of a medium containing the product in methylcyclohexane.

It has now been found that by using particular crystallization conditions, a novel stable crystalline form called form II is obtained.

The crystalline form II of rimonabant has been characterized and compared to the crystalline form I previously described.

The infrared (I.R.) spectra of the 2 crystalline forms of rimonabant have been recorded on Perkin Elmer System 2000 FT-IR spectrophotometers, between 400 cm⁻¹ and 4000 cm⁻¹, with a resolution of 4 cm⁻¹, in a potassium bromide pellet, the test compound being at the concentration of 0.5% by mass.

These spectra are characterized by the absorption bands presented in the following Tables 1 and 2.

TABLE 1
I.R. spectrum, form I
λ (cm⁻¹)

3265.53
1667.78
901.57
761.61

TABLE 2
I.R. spectrum, form II

| λ (cm⁻¹) | λ (cm⁻¹) |
|---|---|
| 3311.30 | 1484.80 |
| 2787.23 | 986.57 |
| 1683.48 | 922.58 |
| 1526.55 | 781.02 |

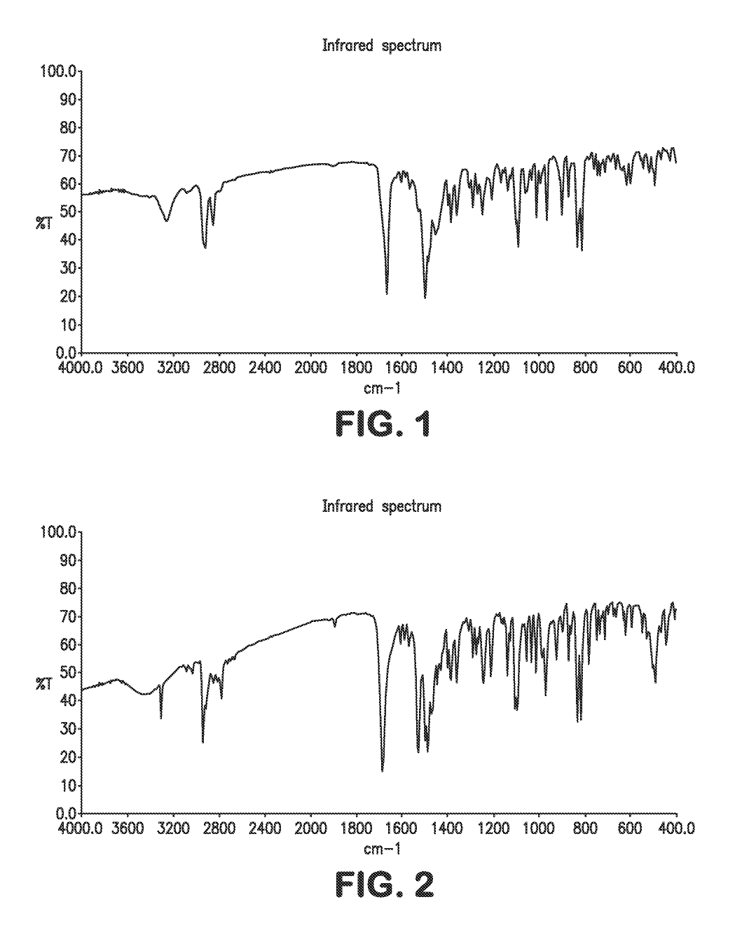

The corresponding spectra are reproduced in FIGS. 1 and 2.

The X-ray (XR) powder diffractograms for the crystalline forms I and II were recorded. The X-ray powder diffraction profile (diffraction angle) was established with a Siemens D500TT (theta/theta), Bragg-Brentano type, diffractometer; CuKα₁ source, λ=1.5406 Å; scanning range 2° to 40° at 1° per minute in Bragg 2 theta.

The characteristic lines of the diffractograms of the 2 compounds are presented in the following tables:

TABLE 3
Powder X-rays, form I

| Peak | Angle |
|---|---|
| ångstroms | 2-Theta° |
| d = 9.65570 | 9.151 |
| d = 7.58833 | 11.652 |
| d = 7.17682 | 12.323 |
| d = 5.51204 | 16.067 |
| d = 5.38190 | 16.458 |
| d = 5.25349 | 16.863 |
| d = 4.82130 | 18.387 |
| d = 4.56563 | 19.426 |
| d = 4.28517 | 20.712 |
| d = 4.16860 | 21.297 |
| d = 3.87660 | 22.922 |
| d = 3.27222 | 27.231 |

TABLE 4
Powder X-ray, form II

| Peak | Angle |
|---|---|
| ångstroms | 2-Theta° |
| d = 17.41664 | 5.070 |
| d = 8.70963 | 10.148 |
| d = 8.19062 | 10.793 |
| d = 5.82785 | 15.191 |
| d = 4.63425 | 19.136 |
| d = 3.49212 | 25.486 |

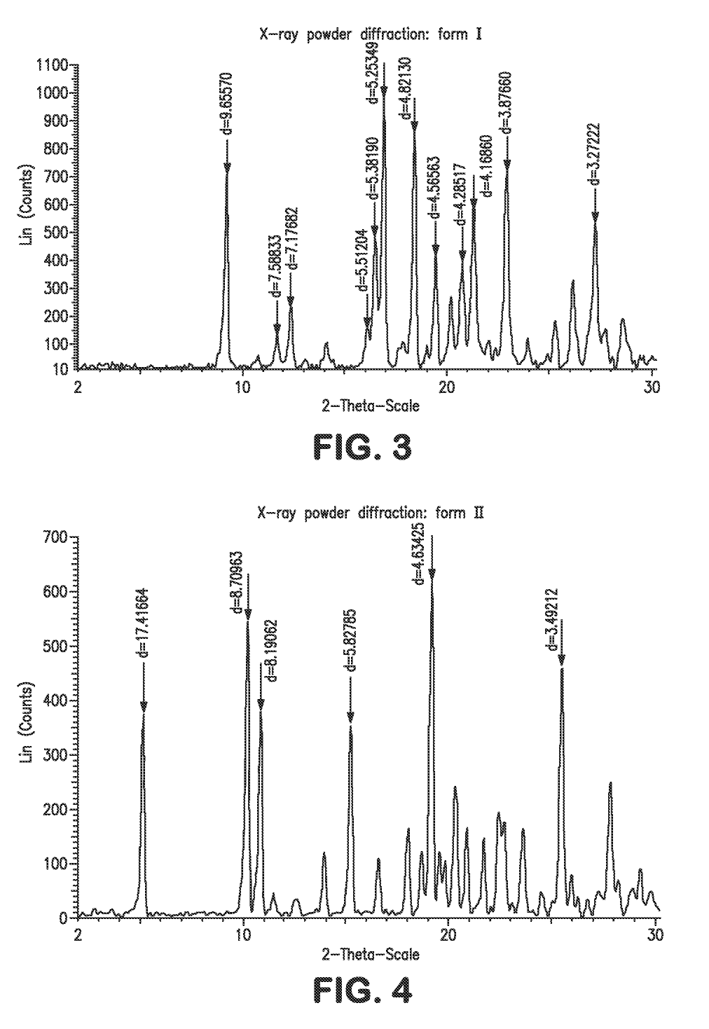

The corresponding diffractograms are reproduced in FIGS. 3 and 4. Rimonabant crystalline form II is also characterized by its crystal structure for which the lattice parameters were determined by single-crystal X-ray diffraction.

TABLE 5
Lattice parameter, form II

| | |
|---|---|
| Molecular formula | C13 N4 O C22 H21 |
| Molecular weight | 463.78 |
| Lattice structure | monoclinic |
| Space group | P 21/c |
| Symmetry elements | ‘x, y, z’ |
| | ‘−x, y + 1/2, −z + 1/2’ |
| | ‘−x, −y, −z’ |
| | ‘x, −y − 1/2, z − 1/ |
| Lattice parameter a | 17.4670(7)Å |
| Lattice parameter b | 9.2820(9)Å |
| Lattice parameter c | 13.9450(14)Å |
| Lattice parameter α | 90.00° |
| Lattice parameter β | 91.994(5)° |
| Lattice parameter γ | 90.00° |
| Lattice volume | 2259.5(3)Å3 |
| Number of molecules per | 4 |
| cell: Z | |

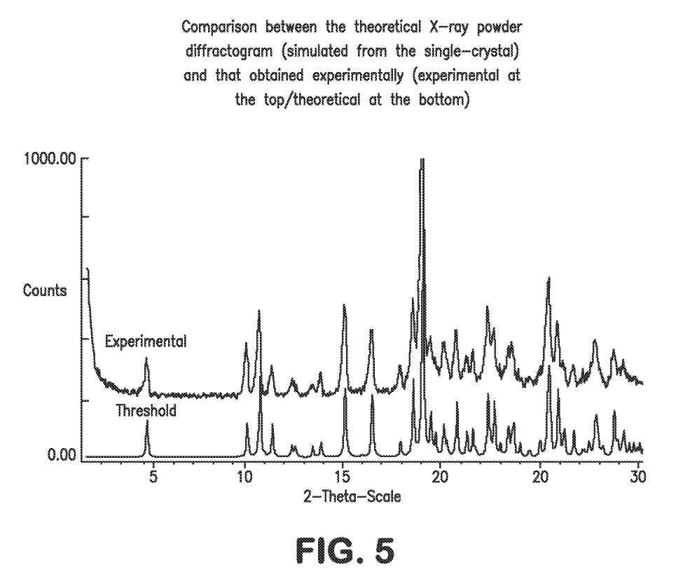

From the rimonabant single-crystal form II, a simulated powder diffractogram (theoretical diffractogram) was obtained which was compared with that obtained experimentally. FIG. 5 shows the comparison of the diffractograms obtained.

The very high similarity observed indicates that the structure contained in the powder corresponds to that determined in the single-crystal and that this structure is unique, that is to say that there is no other polymorphic form mixed with form II of rimonabant.

Differential enthalpic analysis of the 2 crystalline forms was carried out under the same conditions on an MDSC 2920 apparatus for differential enthalpic analysis, marketed by TA Instruments SARL (PARIS); the procedure is carried out under a nitrogen atmosphere, the initial temperature is 30° C., it increases at the rate of 10° C./minute.

For each compound, the melting peak and the difference in enthalpy of the substance (ΔH) is measured before and after melting, in joules per gram of material.

Form I has a melting peak at 156±2° C. with ΔH=65±2 J/g.

Form II has a melting peak at 157±2° C. with ΔH=66±2 J/g.

Thus, the present invention relates to the crystalline polymorph of rimonabant (form II), characterized by infrared spectrum absorption bands as described in Table 2.

This polymorph is also characterized by the characteristic lines of the X-ray powder diffractogram as described in Table 4.

Furthermore, the crystalline polymorph is characterized by a melting peak at 157±2° C. with ΔH=66±2 J/g.

The solubility of the 2 crystalline forms of rimonabant in the same solvent was also measured. The method used is described in Measurement of Solubility in J. W. Mullin. Crystallization: 3rd edition, Ipswich (GB): Butterworth-Heinemann, 1993, p. 105.

The measurements were carried out for each of the crystalline forms, in solution in methylcyclohexane at temperatures varying from 10° C. to 70° C. At equilibrium, for each temperature, the undissolved crystalline form is characterized by infrared spectrography, in particular by its main bands. The 2 trials carried out for each of the crystalline forms are presented in the table below:

TABLE 6
Solubility

Concentration in g/l

| Temperature ° C. | Form I | Form II |
|---|---|---|
| 10.00 | 4.86 | 4.50 |
| 20.00 | 6.92 | 6.60 |
| 30.00 | 9.30 | 9.20 |
| 40.00 | 13.70 | 12.60 |
| 50.00 | 20.40 | 19.00 |
| 60.00 | 31.20 | 29.20 |
| 70.00 | 52.40 | 42.00 |

It is observed that rimonabant form II is less soluble at all the temperatures between 10° C. and 70° C., this shows that rimonabant form II is thermodynamically more stable than rimonabant form I.

According to the present invention, the method for producing the crystalline form II of rimonabant is characterized in that:

a) rimonabant is dissolved in the hot state in a solvent chosen from:
methylcyclohexane in the pure state or containing 1 to 10% of water by volume,
acetonitrile
4-methyl-2-pentanone,
acetone,
or a mixture of these solvents;

b) where appropriate, the medium is cooled to a temperature of between 5° C. and 25° C.,

c) the crystals formed are filtered at a temperature of between 5° C. and 25° C.

According to a particular embodiment, which is the subject of the present invention, at the end of step a), the medium is inoculated with rimonabant having the crystalline form II.

The rimonabant which is dissolved in step a) is rimonabant in the crystalline form I as obtained according to patent EP 0 656 354 or rimonabant form II or a mixture of the two forms. It is also possible to prepare rimonabant in crystalline form I directly from 5-(4-chlorophenyl)-1-(2,4-dichlorophenyl)-4-methylpyrazole-3-carboxylic acid, according to the method described in EP 0 656 354; the acid is converted to its acid chloride by the action of thionyl chloride, and then 1-aminopiperidine is caused to react in the presence of triethylamine.

The present invention has several particular embodiments.

One particular method is characterized in that:

a) rimonabant is dissolved at the concentration of 150 to 220 g/l by heating to the reflux temperature of a solvent consisting of methylcyclohexane containing 1 to 10% of water, and then either steps b), c) and d) below are carried out, or steps c) and d) are carried out directly;

b) the medium is cooled to a temperature of between 40° C. and 50° C., and then the medium is heated to a temperature of between 60° C. and 75° C. and maintained for 2 hours;

c) the temperature is reduced with a cooling step of −15° C. to −20° C. per hour up to a temperature of between 5° C. and 20° C.;

d) the crystals formed are filtered at a temperature of between 5° C. and 20° C.

Preferably, this method is characterized in that:
in step a), the compound is dissolved at the concentration of 200 g/l in a solvent consisting of methylcyclohexane containing 1 to 5% of water, by heating to the reflux temperature of the solvent;
in step b), the medium is cooled to 45° C. over 30 minutes, and then the medium is heated to 70° C.±2° C. and the temperature is maintained for 2 hours;
in step c) the temperature is reduced with a step of −15° C. to −20° C. per hour up to a temperature of between 15° C. and 20° C.

According to one variant of the method according to the invention:

a) rimonabant is dissolved at the concentration of 50 to 250 g/l in a solvent consisting of methylcyclohexane in the pure state or containing 1 to 10% of water;

b) the medium is cooled to a temperature of between 65° C. and 75° C. and allowed to stand for 2 hours at this temperature;

c) the medium is inoculated by addition of 1% to 5% by weight of rimonabant, crystalline form II;

d) the temperature is reduced with a cooling step of −15° C. to −20° C. per hour up to a temperature of between 10° C. and 20° C.;

e) the crystals formed are filtered at a temperature of between 10° C. and 20° C.

Preferably, this method is characterized in that:
in step a), rimonabant is at the concentration of 120 to 150 g/1;
in step b), the mixture is cooled to 70° C.;
in step c), the crystallization is initiated with 2% by weight of rimonabant in crystalline form II.

According to another method of preparation:

a) rimonabant is dissolved at the concentration of 200 to 250 g/l while heating to the temperature of the solvent consisting either of methylcyclohexane, or of methyl isobutyl ketone, or of acetone, or of the mixture of these solvents;

b) the temperature is reduced with a cooling step of −10° C. to −20° C. per hour until the nucleation begins, optionally the nucleating temperature is maintained for 1 hour;

c) the temperature is again reduced with a cooling step of −10° C. to −20° C. per hour until a temperature of between 10° C. and 20° C. is obtained;

d) the crystals are filtered at a temperature of between 10° C. and 20° C.

Another embodiment of the method according to the invention is characterized in that:

a) rimonabant is dissolved at the concentration of 120 to 250 g/l by heating at the reflux temperature of the solvent which is methylcyclohexane;

b) the mixture is cooled to a temperature of between 80° C. and 90° C.;

c) the medium is inoculated by adding 1% to 5% by weight of rimonabant in crystalline form II in suspension in methylcyclohexane and the temperature is maintained for one hour between 80° C. and 90° C.;

d) the temperature is reduced with a cooling step of −15° C. to −20° C. per hour up to a temperature of between 10° C. and 20° C.;

e) the crystals formed are filtered at a temperature of between 10° C. and 20° C.

Preferably, this method is characterized in that:
in step a), rimonabant is dissolved at the concentration of 200 g/l in the solvent;
in step b), the mixture is cooled to 85° C.±2° C.;
in step c), the mixture is inoculated with 2% by weight of rimonabant form II, and then the temperature of the medium is maintained for one hour at 85° C.±2° C.

Another particular method of production according to the invention is characterized in that:

a) rimonabant is dissolved at room temperature in acetonitrile, to saturation;

b) the mixture is left to evaporate at room temperature;

c) the crystals formed are recovered.

According to another embodiment, it is possible to use a solvent which is not very polar, such as pure methylcyclohexane and to obtain the rimonabant in form II using a seed crystal of rimonabant form II for the crystallization.

This method of preparing the compound according to the invention is characterized in that:

a) rimonabant at the concentration of 150 g/l to 300 g/l in methylcyclohexane is heated to a temperature of between 85° C. and 95° C.;

b) the medium is inoculated with 1% to 5% by weight of rimonabant in crystalline form II and the temperature is maintained between 85° C. and 95° C. for several hours until form I disappears;

c) the temperature is reduced with a cooling step of −10° C. to −20° C. per hour up to a temperature of 10° C. to 20° C.;

d) the crystals formed are filtered at a temperature of between 10° C. and 20° C.

According to a particular embodiment, in step a), rimonabant is prepared at the concentration of 150 g/l to 300 g/l in methylcyclohexane by treating 5-(4-chlorophenyl)-1-(2,4-dichlorophenyl)-4-methylpyrazole-3-carboxylic acid chloride with 1-aminopiperidine in a mixture of methylcyclohexane and tetrahydrofuran in the presence of triethylamine.

The crystalline form II of rimonabant has a stability greater than that of form I described above. Furthermore, the crystalline form II of rimonabant may be obtained in a specific manner by means of the method of the invention; this constitutes an advantage for the industrial manufacture of the crystalline form II of rimonabant.

Thus, the crystalline form II of rimonabant is particularly suitable for the manufacture of pharmaceutical compositions useful for treating any disease in which an antagonist of the CB₁ cannabinoid receptors is involved.

According to one of its aspects, the subject of the present invention is pharmaceutical compositions containing, as active ingredient, rimonabant in crystalline form II.

In the pharmaceutical compositions of the present invention for administration by the oral, sublingual, subcutaneous, intramuscular, intravenous, transdermal or local route, the active ingredient, alone or in combination with another active ingredient, can be administered in single-dose administration forms, as a mixture with conventional pharmaceutical vehicles, to animals and human beings. The appropriate single-dose administration forms comprise the forms by the oral route, such as tablets, gelatin capsules, pills, powders, granules and oral solutions or suspensions, sublingual and buccal administration forms, aerosols, implants, local, transdermal, subcutaneous, intramuscular, intravenous, intranasal or intraocular administration forms.

In the pharmaceutical compositions of the present invention, the active ingredient or active ingredients are generally formulated as dosage units. The dosage unit contains 0.5 to 300 mg, advantageously from 5 to 60 mg, preferably from 5 to 40 mg per dosage unit, for daily administrations, once or several times per day.

Although these dosages are examples of average situations, there may be specific cases where higher or lower dosages are appropriate; such dosages also form part of the invention. According to the usual practice, the dosage appropriate for each patient is determined by the doctor according to the method of administration and the age, the weight and the response of the said patient.

When a solid composition is prepared in the form of tablets or gelatin capsules, a mixture of pharmaceutical excipients is added to the micronized or nonmicronized active ingredients, which mixture can be composed of diluents, such as, for example, lactose, mannitol, microcrystalline cellulose, starch or dicalcium phosphate, of binders, such as, for example, polyvinylpyrrolidone or hydroxypropylmethylcellulose, of disintegrating agents, such as crosslinked polyvinylpyrrolidone or crosslinked carboxymethylcellulose, croscarmellose sodium, of flow agents, such as silica or talc, or of lubricants, such as magnesium stearate, stearic acid, glyceryl tribehenate or sodium stearylfumarate.

Wetting agents or surfactants, such as sodium lauryl sulphate, polysorbate 80 or poloxamer 188, can be added to the formulation.

The tablets can be prepared by various techniques: direct tableting, dry granulation, wet granulation or hot-melt.

The tablets can be bare or sugar-coated (with sucrose, for example) or coated with various polymers or other appropriate materials.

The tablets can have a flash, delayed or sustained release by preparing polymeric matrices or by using specific polymers when forming the thin film.

The gelatin capsules may be soft or hard and may or may not be coated with a thin film, so as to have a flash, sustained or delayed activity (for example via an enteric form). They can comprise not only a solid formulation formulated as above for tablets but also liquids or semi-solids.

A preparation in the form of a syrup or elixir can comprise the active ingredient or active ingredients in conjunction with a sweetener, preferably a calorie-free sweetener, methylparaben and propylparaben, as antiseptic, as well as a flavouring agent and an appropriate colorant.

The water-dispersible powders or granules can comprise the active ingredient or active ingredients as a mixture with dispersing agents, wetting agents or suspending agents, such as polyvinylpyrrolidone or polyvidone, as well as with sweeteners or taste corrigents.

For rectal administration, recourse is had to suppositories which are prepared with binders which melt at the rectal temperature, for example cocoa butter or polyethylene glycols.

For parenteral, intranasal or intraocular administration, use is made of aqueous suspensions, isotonic saline solutions or sterile and injectable solutions which comprise pharmacologically compatible dispersing agents and/or solubilizing agents, for example propylene glycol or butylene glycol.

Thus, to prepare an aqueous solution which can be injected by the intravenous route, use may be made of a cosolvent, such as, for example, an alcohol, such as ethanol, or a glycol, such as polyethylene glycol or propylene glycol, and of a hydrophilic surfactant, such as polysorbate 80 or poloxamer 188. To prepare an oily solution which can be injected by the intramuscular route, the active ingredient can be dissolved with a triglyceride or a glyceryl ester.

For local administration, use may be made of creams, ointments, gels, eyewashes or sprays.

For transdermal administration, use may be made of patches in multilaminar or reservoir form, in which the active ingredient can be in alcoholic solution.

For administration by inhalation, use is made of an aerosol comprising, for example, sorbitan trioleate or oleic acid and trichlorofluoromethane, dichlorofluoromethane, dichlorotetrafluoroethane, freon substitutes or any other biologically compatible propellant gas; use may also be made of a system comprising the active ingredient, alone or in combination with an excipient, in powder form.

The active ingredient or active ingredients can also be presented in the form of a complex with a cyclodextrin, for example α-, β- or γ-cyclodextrin or 2-hydroxypropyl-β-cyclodextrin or methyl-β-cyclodextrin.

The active ingredient or active ingredients can also be formulated in the form of microcapsules or microspheres, optionally with one or more vehicles or additives.

Use may be made of implants among the sustained-release forms of use in the case of chronic treatments. These implants can be prepared in the form of an oily suspension or in the form of a suspension of microspheres in an isotonic medium.

Preferably, rimonabant in crystalline form II is administered by the oral route, as a single dose per day.

According to another of its aspects, the invention also relates to a method which consists in administering a therapeutically effective quantity of rimonabant in crystalline form II.

### EXAMPLE 1

### Production of the Form II With No Seed Crystal in Methylcyclohexane Containing 1.64% Water

40 g of N-piperidino-5-(4-chlorophenyl)-1-(2,4-dichlorophenyl)-4-methylpyrazole-3-carboxamide are solubilized, at room temperature, in 80 ml of tetrahydrofuran and 240 ml of methylcyclohexane. The tetrahydrofuran is carried away by distillation at atmospheric pressure. The heating is then interrupted and when the temperature is 80° C.±5° C., 4 ml of deionized water are added. After cooling to 45° C.±3° C. and maintaining for at least 30 minutes, the product crystallizes. The heterogeneous medium is then heated again at 70° C.±2° C. for a period of at least 2 hours. The crystallization of the form II is completed by cooling to 20° C.±3° C. The crystals formed are filtered, washed with methylcyclohexane and dried under vacuum at 75° C.

In this trial, 38 g of form II of rimonabant are obtained.

### EXAMPLE 2

### Production of Form II in Methylcyclohexane Containing 1.42% Water With 2% Seed Crystal of Form II

350 ml of methylcyclohexane and 5 ml of deionized water are added to 50 g of N-piperidino-5-(4-chlorophenyl)-1-(2,4-dichlorophenyl)-4-methylpyrazole-3-carboxamide. The reaction medium is heated to reflux temperature and then the heating is interrupted. At 70° C.±3° C., the crystallization is initiated by addition of 1 gram of substance of form II. The mixture is thus stirred for 2 hours at 70° C. and then cooled to 20° C.±3° C. The crystals formed are filtered, washed with methylcyclohexane and dried under vacuum at 75° C.

In this trial, 47.6 g of form II of rimonabant were obtained.

### EXAMPLE 3

### Production of Form II in Pure 4-methyl-2-pentanone

50 ml of 4-methyl-2-pentanone are added to 10 g of N-piperidino-5-(4-chlorophenyl)-1-(2,4-dichlorophenyl)-4-methylpyrazole-3-carboxamide.

The reaction medium is heated to reflux temperature in order to obtain homogenization and then cooled to 20° C.±3° C. The expected product crystallizes. The crystals formed are filtered, washed with the minimum necessary volume of 4-methyl-2-pentanone and dried under vacuum at 60° C.

In this trial, 4 g of form II of rimonabant were obtained.

### EXAMPLE 4

### Production of Form II in a 20% 4-methyl-2-pentanone and 80% Methylcyclohexane Mixture

10 ml of 4-methyl-2-pentanone and 40 ml of methylcyclohexane are added to 10 g of N-piperidino-5-(4-chlorophenyl)-1-(2,4-dichlorophenyl)-4-methylpyrazole-3-carboxamide.

The reaction medium is heated to reflux temperature in order to obtain homogenization. The heating is interrupted and the crystallization of the expected product is then observed at around 40° C. and then the mixture is kept stirred at 20° C.±3° C. The crystals formed are filtered, drained and dried under vacuum at 60° C.

In this trial, 7.9 grams of form II of rimonabant were obtained.

### EXAMPLE 5

### Production of Form II in a 60% 4-methyl-2-pentanone and 40% Methylcyclohexane mixture

30 ml of 4-methyl-2-pentanone and 20 ml of methylcyclohexane are added to 10 g of N-piperidino-5-(4-chlorophenyl)-1-(2,4-dichlorophenyl)-4-methylpyrazole-3-carboxamide.

The reaction medium is heated to reflux temperature; homogenization of the medium is thus obtained. The heating is then interrupted and then the mixture is cooled to 20° C.±3° C. The expected product crystallizes. The crystals formed are filtered, drained and then dried under vacuum at 60° C.

In this trial, 4.8 g of form II of rimonabant were obtained.

### EXAMPLE 6

### Production of Form II in an 80% 4-methyl-2-pentanone and 20% Methylcyclohexane Mixture

40 ml of methyl-4-pentanone and 10 ml of methylcyclohexane are added to 10 g of N-piperidino-5-(4-chlorophenyl)-1-(2,4-dichlorophenyl)-4-methylpyrazole-3-carboxamide.

Homogenization of the reaction medium is obtained at the reflux temperature of the solvent. The heating is then interrupted and the medium is allowed to return to 20° C.±3° C. The expected product crystallizes. The crystals formed are filtered, drained and then dried under vacuum at 60° C.

In this trial, 4 g of form II of rimonabant were obtained.

### EXAMPLE 7

### Production of Form II with 2% Seed Crystal of Form II From 5-(4-chlorophenyl)-1-(2,4-dichlorophenyl)-4-methylpyrazole-3-carboxylic Acid, in Methylcyclohexane

A solution of 72.2 g of thionyl chloride in 60 ml of methylcyclohexane is added, after heating to 83° C.±3° C., under a nitrogen atmosphere, to a suspension of 190.80 g of 5-(4-chlorophenyl)-1-(2,4-dichlorophenyl)-4-methylpyrazole-3-carboxylic acid in 940 ml of methylcyclohexane.

The mixture is stirred for 2 hours at 83° C.±3° C. and then the temperature of the reaction medium is raised over 1 hour up to the reflux temperature of the methylcyclohexane while removing the excess of thionyl chloride by distillation. The reaction medium is cooled to room temperature and a solution of 7 ml of triethylamine in 382 ml of tetrahydrofuran is added.

The solution obtained is added over 15 minutes at 12° C.±3° C. to a medium composed of 50.08 g of triethylamine, 55.10 g of 1-aminopiperidine and 460 ml of methylcyclohexane. The temperature is allowed to rise to 20° C.±5° C. and then the organic phase is successively washed at 70° C.±3° C. with deionized water and acetic acid at 4% in water. The washes of the organic phase at 70° C.±3° C. are completed with a 1.5% NaOH solution and then with deionized water and the tetrahydrofuran and the water are carried away by azeotropic distillation at atmospheric pressure. The heating is then interrupted and when the temperature is 85° C., the crystallization of the expected products is initiated by adding 4 g of substance of form II. The mixture is thus stirred for 1 hour at 85° C.±3° C. and then cooled to 10° C.±3° C. over 5 hours and maintained for 2 hours at 10° C. The crystals formed are filtered, washed with methylcyclohexane, and dried under vacuum at 60° C.

In this trial, 217 g of form II of rimonabant were obtained.

What is claimed is:

1. Crystalline form II of rimonabant which exhibits the infrared spectrum absorption bands described below:

| λ (cm⁻¹) | λ (cm⁻¹) |
|---|---|
| 3311.30 | 1484.80 |
| 2787.23 | 986.57 |
| 1683.48 | 922.58 |
| 1526.55 | 781.02 |

2. The crystalline form II of rimonabant according to claim 1, which exhibits the X-ray powder diffractogram lines described below:

| Peak | Angle |
|---|---|
| ångstroms | 2-Theta° |
| d = 17.41664 | 5.070 |
| d = 8.70963 | 10.148 |
| d = 8.19062 | 10.793 |
| d = 5.82785 | 15.191 |
| d = 4.63425 | 19.136 |
| d = 3.49212 | 25.486 |

3. The crystalline form II of rimonabant according to claim 1, which exhibits a melting peak at 157±2° C. with ΔH=66±2 J/g.

4. A Method for preparing the compound according to claim 1 comprising:
a) dissolving rimonabant in the hot state in a solvent chosen from:
methylcyclohexane in the pure state or containing 1 to 10% of water by volume, acetonitrile,
4-methyl-2-pentanone, or acetone,
b) cooling, where appropriate, the solution obtained in step a) above to a temperature of between 5° C. and 25° C. to form crystals, and
c) filtering the crystals formed in step b) at a temperature of between 5° C. and 25° C.

5. The method according to claim 4 wherein after step a), the solution is inoculated with rimonabant, crystalline form II.

6. The method according to claim 4 wherein
a) dissolving rimonabant at the concentration of 150 to 220 g/l by heating to the reflux temperature of a solvent consisting of methylcyclohexane containing 1 to 10% of water, and then either steps b), c) and d) below are carried out, or steps c) and d) are carried out directly;
b) cooling the solution to a temperature of between 40° C. and 50° C., and then the solution is heated to a temperature of between 60° C. and 75° C. and maintained for 2 hours;
c) reducing the temperature with a cooling step of −15° C. to −20° C. per hour up to a temperature of between 5° C. and 20° C. to form crystals; and
d) filtering the crystals obtained in step c) at a temperature of between 5° C. and 20° C.

7. The Method according to claim 6 wherein
in step a), dissolving the compound at the concentration of 200 g/l in a solvent consisting of methylcyclohexane containing 1 to 5% of water, by heating to the reflux temperature of the solvent;
in step b), cooling the solution to 45° C. over 30 minutes, and then the solution is heated to 70° C.±2° C. and the temperature is maintained for 2 hours; and
in step c), reducing the temperature with the cooling step of −15° C. to −20° C. per hour up to a temperature of between 15° C. and 20° C.

8. The Method according to claim 4 wherein
a) dissolving rimonabant at the concentration of 50 to 250 g/l in a solvent consisting of methylcyclohexane in the pure state or containing 1 to 10% of water;
b) cooling the solution to a temperature of between 65° C. and 75° C. and allowed to stand for 2 hours at this temperature;
c) inoculating the solution by addition of 1% to 5% by weight of rimonabant, crystalline form II;
d) reducing the temperature with a cooling step of −15° C. to −20° C. per hour up to a temperature of between 10° C. and 20° C. to form crystals; and
e) filtering the crystals formed at a temperature of between 10° C. and 20° C.

9. The method according to claim 8 wherein
in step a), rimonabant is present at the concentration of 120 to 150 g/l;
in step b), the mixture is cooled to 70° C.; and
in step c), the crystallization is initiated with 2% by weight of rimonabant in crystalline form II.

10. The method according to claim 4 wherein
a) dissolving rimonabant at the concentration of 200 to 250 g/l while heating to the temperature of the solvent consisting either of methylcyclohexane, or of methyl isobutyl ketone, or of acetone;
b) reducing the temperature with a cooling step of −10° C. to −20° C. per hour until the nucleation begins, optionally the nucleating temperature is maintained for 1 hour;
c) again reducing the temperature with a cooling step of −10° C. to −20° C. per hour until a temperature of between 10° C. and 20° C. is obtained to form crystals; and
d) filtering the crystals at a temperature of between 10° C. and 20° C.

11. The method according to claim 4 wherein
a) dissolving rimonabant at the concentration of 120 to 250 g/l by heating at the reflux temperature of the solvent which is methylcyclohexane;
b) cooling the mixture to a temperature of between 80° C. and 90° C.;
c) inoculating the medium by adding 1% to 5% by weight of rimonabant in crystalline form II in suspension in methylcyclohexane and the temperature is maintained for one hour between 80° C. and 90° C.;
d) reducing the temperature with a cooling step of −15° C. to −20° C. per hour up to a temperature of between 10° C. and 20° C. to form crystals; and
e) filtering the crystals formed at a temperature of between 10° C. and 20° C.

12. The method according to claim 11 wherein
in step a), rimonabant is dissolved at the concentration of 200 g/l in the solvent;
in step b), the mixture is cooled to 85° C.±2° C.;
in step c), the mixture is inoculated with 2% by weight of rimonabant form II, and then the temperature of the medium is maintained for one hour at 85° C.±2° C.

13. The method according to claim 4 wherein
a) rimonabant is dissolved at room temperature in acetonitrile, to saturation;
b) the mixture is left to evaporate at room temperature; and
c) the crystals formed are recovered.

14. The method for preparing the compound according to claim 1 wherein
a) rimonabant at the concentration of 150 g/l to 300 g/l in methylcyclohexane is heated to a temperature of between 85° C. and 95° C.;
b) the medium is inoculated with 1% to 5% by weight of rimonabant in crystalline form II and the temperature is maintained between 85° C. and 95° C. for several hours until form I disappears;
c) the temperature is reduced with a cooling step of −15° C. to −20° C. per hour up to a temperature of between 10° C. and 20° C.; and
d) the crystals formed are filtered at a temperature of between 10° C. and 20° C.

15. The method according to claim 14 wherein in step a), rimonabant is prepared at the concentration of 150 g/l to 300 g/l in methylcyclohexane by treating 5-(4-chlorophenyl)-1-(2,4-dichlorophenyl)-4-methylpyrazole-3-carboxylic acid chloride in methylcyclohexane with 1-aminopiperidine in a mixture of methylcyclohexane and tetrahydrofuran in the presence of triethylamine.

16. A pharmaceutical composition comprising, as active ingredient, the crystalline form II of rimonabant according to claim 1 in combination with at least one pharmaceutical excipient.

17. A method for treating a disease in which an antagonist of the CB₁ cannabinoid receptor is involved which comprises administering to a patient in need of said treatment an effective amount of a compound according to claim 1.

18. A method for treating a disease in which an antagonist of the CB₁ cannabinoid receptor is involved which comprises administering to a patient in need of said treatment an effective amount of a compound according to claim 2.

19. A method for treating a disease in which an antagonist of the CB₁ cannabinoid receptor is involved which comprises administering to a patient in need of said treatment an effective amount of a compound according to claim 3.

20. A pharmaceutical composition comprising, as active ingredient, the crystalline form II of rimonabant according to claim 2 in combination with at least one pharmaceutical excipient.

21. A Pharmaceutical composition containing, as active ingredient, the crystalline form II of rimonabant according to claim 3 in combination with at least one pharmaceutical excipient.
